(19) United States
(12) Patent Application Publication
(10) Pub. No.: US 2003/0153497 A1
(43) Pub. Date: Aug. 14, 2003

(54) Use of human not1and not1a orphan receptors

(76) Inventors: William John Cairns, Essex (GB); Julie Caroline Holder, Hertfordshire (GB); Lisa Patel, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/221,909

(22) PCT Filed: Mar. 19, 2001
(86) PCT No.: PCT/GB01/01211

(30) Foreign Application Priority Data
Mar. 21, 2000 (GB) 0006864.3

(51) Int. Cl. A61K 48/00; A61K 38/24

(52) U.S. Cl. 514/12; 514/44

(57) ABSTRACT

The use of NOT1 or NOT1 a polypeptides and polynucleotides in the design of protocols for the treatment of obesity, insulin resistance, type 2 diabetes, impaired glucose tolerance, cachexia or liposarcoma among others, and diagnostic assays for such conditions.

### FIELD OF THE INVENTION

This invention relates to new uses for polynucleotides and polypeptides encoded by them, to their use in therapy and in identifying compounds which may be agonists which are potentially useful in therapy.

### SUMMARY OF THE INVENTION

In one aspect, the invention relates to new uses of the orphan nuclear receptor, NOT1 or its splice variant NOT1a polynucleotides and polypeptides. Such uses include the treatment of obesity, insulin resistance, type 2 diabetes, impaired glucose tolerance, cachexia and liposarcoma, hereinafter referred to as “the Diseases”, amongst others In another aspect the invention relates to methods for identifying compounds which activate NOT1 or NOT1a polypeptides, for example agonists, using NOT1 or NOT1a materials, and treating conditions associated with NOT1 or NOT1a imbalance with the identified agonist compounds. In a still further aspect, the invention relates to diagnostic assays for detecting diseases associated with inappropriateNOT1 or NOT1a activity or levels.

### DESCRIPTION OF THE INVENTION

In a first aspect, the present invention relates to the use of a compound selected from:

(a) a NOT1 or NOT1a polypeptide;

(b) a compound which activates a NOT1 or NOT1a polypeptide; or

(c) a polynucleotide encoding a NOT1 or NOT1a polypeptide, for the manufacture of a medicament for treating:

(i) obesity;

(ii) insulin resistance;

(iii) type 2 diabetes;

(iv) impaired glucose tolerance;

(v) cachexia; or

(vi) liposarcoma.

Such NOT1 polypeptides include isolated polypeptides comprising an amino acid sequence which has at least 95% identity, preferably at least 97-99% identity, to that of SEQ ID NO:2 over the entire length of SEQ ID NO:2. Such polypeptides include those comprising the amino acid of SEQ ID NO:2.

Further polypeptides include isolated polypeptides in which the amino acid sequence has at least 95% identity, preferably at least 97-99% identity, to the amino acid sequence of SEQ ID NO:2 over the entire length of SEQ ID NO:2. Such polypeptides include the polypeptide of SEQ ID NO:2. Further peptides include isolated polypeptides encoded by a polynucleotide comprising the sequence contained in SEQ ID NO:1.

NOT1a polypeptides include isolated polypeptides comprising an amino acid sequence which has at least 95% identity, preferably at least 97-99% identity, to that of SEQ ID NO:4 over the entire length of SEQ ID NO:4. Such polypeptides include those comprising the amino acid of SEQ ID NO:4

Further NOT1a polypeptides include isolated polypeptides in which the amino acid sequence has at least 95% identity, preferably at least 97-99% identity, to the amino acid sequence of SEQ ID NO:4 over the entire length of SEQ ID NO:4. Such polypeptides include the polypeptide of SEQ ID NO:4. Further peptides include isolated polypeptides encoded by a polynucleotide comprising the sequence contained in SEQ ID NO:3.

The polypeptides may be in the form of the “mature” protein or may be a part of a larger protein such as a precursor or a fusion protein. It is often advantageous to include an additional amino acid sequence which contains secretory or leader sequences, pro-sequences, sequences which aid in purification such as multiple histidine residues, or an additional sequence for stability during recombinant production.

The NOT1 and NOT1a polypeptides relating to the present invention also include variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

NOT1 and NOT1a polypeptides relating to the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

In a further aspect, the present invention relates to NOT1 or NOT1a polynucleotides. Such polynucleotides include isolated polynucleotides comprising a nucleotide sequence encoding a polypeptide which has at least 95% identity to the amino acid sequence of SEQ ID NO:2 or SEQ ID NO:4, over the entire length of SEQ ID NO:2 or SEQ ID NO:4. In this regard, polypeptides which have at least 97% identity are highly preferred, whilst those with at least 98-99% identity are more highly preferred, and those with at least 99% identity are most highly preferred. Such polynucleotides include a polynucleotide comprising the nucleotide sequence contained in SEQ ID NO:1 or SEQ ID NO:3 encoding the polypeptides of SEQ ID NO:2 and SEQ ID NO:4 respectively.

Further polynucleotides relating to the present invention include isolated polynucleotides comprising a nucleotide sequence that has at least95% identity to a nucleotide sequence encoding a polypeptide of SEQ ID NO:2 or SEQ ID NO:4, over the entire coding region. In this regard, polynucleotides which have at least 97% identity are highly preferred, whilst those with at least 98-99% identity are more highly preferred, and those with at least 99% identity are most highly preferred.

Further polynucleotides include isolated polynucleotides comprising a nucleotide sequence which has at least 95% identity to SEQ ID NO:1 or SEQ ID NO:3 over the entire length of SEQ ID NO:1 or SEQ ID NO:3 respectively. In this regard, polynucleotides which have at least 97% identity are highly preferred, whilst those with at least 98-99% identity are more highly preferred, and those with at least 99% identity are most highly preferred. Such polynucleotides include a polynucleotide comprising the polynucleotide of SEQ ID NO:1 or SEQ ID NO:3 as well as the polynucleotide of SEQ ID NO:1 or SEQ ID NO:3.

The invention also relates to polynucleotides which are complementary to all the above described polynucleotides.

The nucleotide sequence of a human NOT1 cDNA is given in SEQ ID NO:1 (from Mages, H. W. et al. Mol. Endocrinol. 1994; 4(11): 1583-1591, EMBL:X75918). The nucleotide sequence of SEQ ID NO:1 is a cDNA sequence and comprises a polypeptide encoding sequence (nucleotide 318 to 3115) encoding a polypeptide of 598 amino acids, the polypeptide of SEQ ID NO:2. The nucleotide sequence encoding the polypeptide of SEQ ID NO:2 may be identical to the polypeptide encoding sequence contained in SEQ ID NO:1 or it may be a sequence other than the one contained in SEQ ID NO:1, which, as a result of the redundancy (degeneracy) of the genetic code, also encodes the polypeptide of SEQ ID NO:2. The nucleotide sequence of the splice variant NOT1a is given in SEQ ID NO:3 (from Patent Application WO00/77202; SmithKline Beecham) and is a cDNA sequence and comprises a polypeptide encoding sequence (nucleotide 1 to 1368) encoding a polypeptide of 455 amino acids, the polypeptide of SEQ ID NO:4., representing a splice variant of NOT1. The nucleotide sequence encoding the polypeptide of SEQ ID NO:4 may be identical to the polypeptide encoding sequence contained in SEQ ID NO:3 or it may be a sequence other than the one contained in SEQ ID NO:3, which, as a result of the redundancy (degeneracy) of the genetic code, also encodes the polypeptide of SEQ ID NO:4.

The gene encoding the NOT1 polypeptide of SEQ ID NO:2 has been localised to human chromosome 2q22-2q23..

Preferred polypeptides and polynucleotides are expected to have, inter alia, similar biological functions/properties to their homologous polypeptides and polynucleotides. Furthermore, preferred polypeptides and polynucleotides of the present invention have at least one NOT1 or NOT1a activity, which may include antigenic activity.

NOT1 and NOT1a polynucleotides may be obtained, using standard cloning and screening techniques, from a cDNA library derived from mRNA in cells of human brain (Sambrook et al., Molecular Cloning: A Laboratory Manual, 2nd Ed., Cold Spring Harbor Laboratory Press, Cold Spring Harbor, N.Y. (1989)). Such polynucleotides can also be obtained from natural sources such as genomic DNA libraries or can be synthesized using well known and commercially available techniques.

When NOT1 or NOT1a polynucleotides are used for the recombinant production of NOT1 or NOT1a polypeptides, the polynucleotide may include the coding sequence for the mature polypeptide, by itself; or the coding sequence for the mature polypeptide in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded for example a hexa-histidine peptide, as provided in the pQE vector (Qiagen, Inc.) and described in Gentz et al., Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Recombinant NOT1 or NOT1a polypeptides may be prepared by processes well known in the art from genetically engineered host cells comprising expression systems. Cell-free translation systems can also be employed to produce such proteins using RNAs derived from NOT1 or NOT1a DNA constructs.

For recombinant production, host cells can be genetically engineered to incorporate expression systems or portions thereof for NOT1 or NOT1a polynucleotides. Introduction of polynucleotides into host cells can be effected by methods described in many standard laboratory manuals, such as Davis et al, Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Preferred such methods include, for instance, calcium phosphate transfection, DEAE-dextran mediated transfection, microinjection, cationic lipid-mediated transfection, electroporation, transduction, scrape loading, ballistic introduction or infection.

Representative examples of appropriate hosts include bacterial cells, such as E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK and HEK 293.

The appropriate nucleotide sequence may be inserted into an expression vector by any of a variety of well-known and routine techniques, such as, for example, those set forth in Sambrook et al., Molecular Cloning, A Laboratory Manual (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals. If the polypeptide is secreted into the medium, the medium can be recovered in order to recover and purify the polypeptide. If produced intracellularly, the cells must first be lysed before the polypeptide is recovered.

NOT1 or NOT1a polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

This invention also relates to the use of polynucleotides of the present invention as diagnostic reagents. Detection of a mutated form of the gene characterised by the polynucleotide of SEQ ID NO:1 or SEQ ID NO:3 which is associated with a dysfunction will provide a diagnostic tool that can add to, or define, a diagnosis of a disease, or susceptibility to a disease, which results from under-expression or otherwise altered expression of the gene. Individuals carrying mutations in the gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeledNOT1 or NOT1a nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (ee, e.g., Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401). In another embodiment, an array of oligonucleotides probes comprising NOT1 or NOT1a nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M.Chee et al., Science, Vol 274, pp 610-613 (1996)).

The diagnostic assays offer a process for diagnosing or determining a susceptibility to the Diseases through detection of mutation in the NOT1 gene by the methods described. In addition, such diseases may be diagnosed by methods comprising determining from a sample derived from a subject an abnormally decreased level of polypeptide or mRNA. Decreased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a NOT1 or NOT1a protein in a sample derived from a host are well-known to those of skill in the art. Such assay methods include radioimmunoassays, competitive-binding assays, Western Blot analysis and ELISA assays.

Thus in another aspect, the present invention relates to a diagonostic kit which comprises:

(a) a NOT1 or NOT1a polynucleotide, preferably the nucleotide sequence of SEQ ID NO:1 or SEQ ID NO:3, or fragments thereof;

(b) a nucleotide sequence complementary to that of (a);

(c) a NOT1 or NOT1a polypeptide, preferably the polypeptide of SEQ ID NO:2 or SEQ ID NO:4, or a fragment thereof; or

(d) an antibody to a NOT1 or NOT1a polypeptide, preferably to the polypeptide of SEQ ID NO:2 or SEQ ID NO:4.

It will be appreciated that in any such kit, (a), (b), (c) or (d) may comprise a substantial component. Such a kit will be of use in diagnosing a disease or suspectability to a disease, particularly obesity, insulin resistance, type 2 diabetes, impaired glucose tolerance,cachexia and liposarcoma, amongst others.

The polypeptides of the invention or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Antibodies generated against NOT1 or NOT1a polypeptides may be obtained by administering the polypeptides or epitope-bearing fragments, analogs or cells to an animal, preferably a non-human animal, using routine protocols. For preparation of monoclonal antibodies, any technique which provides antibodies produced by continuous cell line cultures can be used. Examples include the hybridonia technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

In a further aspect, the present invention relates to genetically engineered soluble fusion proteins comprising a NOT1 or NOT1a polypeptide, or a fragment thereof, and various portions of the constant regions of heavy or light chains of immunoglobulins of various subclasses (IgG, IgM, IgA, IgE). Preferred as an immunoglobulin is the constant part of the heavy chain of human IgG, particularly IgG1, where fusion takes place at the hinge region. In a particular embodiment, the Fc part can be removed simply by incorporation of a cleavage sequence which can be cleaved with blood clotting factor Xa. Furthermore, this invention relates to processes for the preparation of these fusion proteins by genetic engineering, and to the use thereof for drug screening, diagnosis and therapy. A further aspect of the invention also relates to polynucleotides encoding such fusion proteins. Examples of fusion protein technology can be found in International Patent Application Nos. WO94/29458 and WO94/22914.

In a further aspect, the present invention provides for a method of screening compounds to identify those which stimulate the function of NOT1 or NOT1a polypeptides. In general, agonists may be employed for therapeutic and prophylactic purposes for such Diseases as hereinbefore mentioned. Compounds may be identified from a variety of sources, for example, cells, cell-free preparations, chemical libraries, and natural product mixtures. Such agonists so-identified may be natural or modified substrates, ligands, receptors, enzymes, etc., as the case may be, of the polypeptide; or may be structural or functional mimetics thereof (see Coligan et al., Current Protocols in Immunology 1(2):Chapter 5 (1991)).

The screening method may simply measure the binding of a candidate compound to the polypeptide, or to cells or membranes bearing the polypeptide, or a fusion protein thereof by means of a label directly or indirectly associated with the candidate compound. Alternatively, the screening method may involve competition with a labeled competitor. Further, these screening methods may test whether the candidate compound results in a signal generated by activation or inhibition of the polypeptide, using detection systems appropriate to the cells bearing the polypeptide. Further, the screening methods may simply comprise the steps of mixing a candidate compound with a solution containing a polypeptide of the present invention, to form a mixture, measuring NOT1 or NOT1a activity in the mixture, and comparing the NOT1 or NOT1a activity of the mixture to a standard.

NOT1 or NOT1a polynucleotides, polypeptides and antibodies to the polypeptide as hereinabove described may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may enhance the production of polypeptide, for example agonists, from suitably manipulated cells or tissues.

The NOT1 or NOT1a polypeptide may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists of NOT1 or NOT1a polypeptides which compete with the binding of the polypeptide to its receptors, if any. Standard methods for conducting such assays are well understood in the art.

Thus, in another aspect, the present invention relates to a screening kit for identifying agonists, ligands, receptors, substrates, enzymes, etc. for polypeptides of the present invention; or compounds which enhance the production of such polypeptides, which comprises:

(a) a NOT1 or NOT1a polypeptide;

(b) a recombinant cell expressing a NOT1 or NOT1a polypeptide;

(c) a cell membrane expressing a NOT1 or NOT1a polypeptide; or

(d) antibody to a NOT1 or NOT1a polypeptide;

which NOT1 or NOT1a polypeptide is preferably that of SEQ ID NO:2 or SEQ ID NO:4.

It will be appreciated that in any such kit, (a), (b), (c) or (d) may comprise a substantial component.

It will be readily appreciated by the skilled artisan that a polypeptide of the present invention may also be used in a method for the structure-based design of an agonist of the NOT1 or NOT1a polypeptide, by:

(a) determining in the first instance the three-dimensional structure of the NOT1 or NOT1a polypeptide;

(b) deducing the three-dimensional structure for the likely reactive or binding site(s) of an agonist;

(c) synthesing candidate compounds that are predicted to bind to or react with the deduced binding or reactive site; and

(d) testing whether the candidate compounds are indeed agonists.

It will be further appreciated that this will normally be an interative process.

In a further aspect, the present invention provides methods of treating abnormal conditions such as, for instance, obesity, insulin resistance, type 2 diabetes, impaired glucose tolerance, cachexia, or liposarcoma, related to an under-expression of, NOT1 or NOT1a polypeptide activity.

For treating abnormal conditions related to an under-expression ofNOT1 or NOT1a polypeptides and their activity, several approaches are also available. One approach comprises administering to a subject a therapeutically effective amount of a compound which activatesa polypeptide of the present invention, i.e., an agonist as described above, in combination with a pharmaceutically acceptable carrier, to thereby alleviate the abnormal condition. Alternatively, gene therapy may be employed to effect the endogenous production ofNOT1 or NOT1a by the relevant cells in the subject. For example, a NOT1 or NOT1a polynucleotide may be engineered for expression in a replication defective retroviral vector, as discussed above. The retroviral expression construct may then be isolated and introduced into a packaging cell transduced with a retroviral plasmid vector containing RNA encoding a NOT1 or NOT1a polypeptide such that the packaging cell now produces infectious viral particles containing the NOT1 or NOT1a gene. These producer cells may be administered to a subject for engineering cells in vivo and expression of the NOT1 or NOT1a polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a NOT1 or NOT1a polypeptide, such as the soluble form of a NOT1 or NOT1a polypeptide, agonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. NOT1 or NOT1a polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a NOT1 or NOT1a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

NOT1 or NOT1a polypeptides used in treatment can also be generated endogenously in the subject, in treatment modalities often referred to as“gene therapy” as described above. Thus, for example, cells from a subject may be engineered with a NOT1 or NOT1a polynucleotide, such as a DNA or RNA, to encode a NOT1 or NOT1a polypeptide ex vivo, and for example, by the use of a retroviral plasmid vector. The cells are then introduced into the subject.

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1 or SEQ ID NO:3, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 or SEQ ID NO:3 by the numerical percent of the respective percent identity(divided by 100) and subtracting that product from said total number of nucleotides in SEQ ID NO:1 or SEQ ID NO:3, or:

nₙ≦xₙ−(xₙ·y)

wherein nₙ is the number of nucleotide alterations, xₙ is the total number of nucleotides in SEQ ID NO:1 or SEQ ID NO:3, and y is, for instance, 0.70 for 70%, 0.80 for 80%, 0.85 for 85%, 0.90 for 90%, 0.95 for 95%,etc., and wherein any non-integer product of xₙ and y is rounded down to the nearest integer prior to subtracting it from xₙ. Alterations of a polynucleotide sequence encoding the polypeptide of SEQ ID NO:2 or SEQ ID NO:4 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2 or SEQ ID NO:4, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 or SEQ ID NO:4 by the numerical percent of the respective percent identity(divided by 100) and then subtracting that product from said total number of amino acids in SEQ ID NO:2 or SEQ ID NO:4, or:

nₐ≦xₐ−(xₐ·y),

wherein nₐ is the number of amino acid alterations, xₐ is the total number of amino acids in SEQ ID NO:2 or SEQ ID NO:4, and y is, for instance 0.70 for 70%, 0.80 for 80%, 0.85 for 85% etc., and wherein any non-integer product of xₐ and y is rounded down to the nearest integer prior to subtracting it from xₐ.

“Homolog” is a generic term used in the art to indicate a polynucleotide or polypeptide sequence possessing a high degree of sequence relatedness to a subject sequence. Such relatedness may be quantified by determining the degree of identity and/or similarity between the sequences being compared as hereinbefore described. Falling within this generic term are the terms “ortholog”, meaning a polynucleotide or polypeptide that is the functional equivalent of a polynucleotide or polypeptide in another species, and “paralog” meaning a functionally similar sequence when considered within the same species.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications, including but not limited to patents and patent applications, cited in this specification are herein incorporated by reference as if each individual publication were specifically and individually indicated to be incorporated by reference herein as though fully set forth.

### EXAMPLES

### Example 1

Analysis of NOT1 and NOT1a Expression in Human Tissues

Generation of samples for TaqMan mRNA analysis: Human tissue or RNA was purchased (Biochain, San Leandro, Calif.; Invitrogen, Leek, The Netherlands; Clontech, Palo Alto, Calif.) or donated (Netherlands Brain Bank, Amsterdam, the Netherlands) and poly A+ RNA was prepared by the PolyATract method according to manufacturers instructions (Promega, USA). The poly A+ RNA samples from 20 body tissues and 19 brain-regions from 4 individuals per tissue (two males/two females) were quantitated using OD260 nm measurement or the RiboGreen fluorescent method (Molecular Probes, Oregon, USA) and 1 ug of each RNA was reverse transcribed using random nonomers and Superscript II reverse transcriptase according to manufacturers instructions (Life Technologies). The cDNA prepared was diluted to produce up to 1,000 replicate 96-well plates using Biomek robotics (Beckman Coulter, High Wycombe, UK), so that each of the wells contained the cDNA produced from 1 ng RNA for the appropriate tissue. The 96-well plates were stored at −80° C. prior to use.

TaqMan PCR: This was performed following the procedure published by Sarau H. M. et al, (“Identification, Molecular Cloning, Expression and Characterisation of a Cysteinyl Leukotriene Receptor”, Molecular Pharmacology, 1999, 56, 657-663.) TaqMan quantitative PCR was conducted to measure either NOT-1 or NOT1a using replicate 96-well plates. A 20 ul volume of a PCR master mix (containing 2.5 ul TaqMan buffer, 6 ul 25 mM MgCl2, 0.5 ul of 10 mM dATP, 0.5 ul of 20 mM dUTP,0.5 ul of 10 mM dCTP, 0.5 ul of 10 mM dGTP, 0.25 ul Uracil-N-glycosylase, 1 ul of 10 uM forward primer, 1 ul of 10 uM reverse primer, 0.5 ul 5 uM TaqMan probe, 0.125 ul TaqGold [PE Biosystems], 6.625 ul water) was added to each well using Biomek robotics (Beckman Coulter, High Wycombe, UK), and the plate capped using optical caps (PE Biosystems). The PCR reaction was carried out on an ABI7700 Sequence Detector (PE Biosystems) using the PCR parameters: 50° C. for 2 minutes, 95° C. for 10 minutes and 45 cycles of 94° C. for 15 seconds,60° C. for 1 minute, and the level of mRNA-derived cDNA in each sample was calculated from the TaqMan signal using plasmid/genomic DNA calibration standards included in each run. The level of genomic DNA contaminating the original RNA samples was shown to be negligible (<10 copies genomic DNA/ng RNA) by TaqMan measurement of genomic sequence for ten genes in replicate samples taken through the reverse transcription procedure described with the ommission of reverse transcriptase. Gene-specific reagents for NOT-1:

forward primer: 5′-CCGCCAGCAATAATTGACAA-3′,

reverse primer: 5′-TTCCATTATCATTCCAGTTCCTTTG-3′,

TaqMan probe: 5′-CACTTTACCTTTCTAAGACCTCCTCCCAAGCA-3′;

and for NOT1a:

forward primer: 5′-TCCTTCGATTAGCATACAGAATA-3′,

reverse primer: 5′-CCCGTGTCTCTCTGTGACCAT-3′,

TaqMan probe:5′-TCTGCCTTCTCCTGCATTGCTGCC-3′.

Results

The results are shown in Table 1. Analysis of expression of both NOT1 and NOT1a mRNA by TaqMan showed relatively widespread distribution. However, the expression levels found in adipose tissue were significantly higher than for those found in any other tissue. Levels of expression are scored from +++++ to −, with +++++ representing the highest expression levels. +/− indicates weakly detectable expression.

TABLE 1

| | NOT1 | NOT1a |
|---|---|---|
| Brain | ++ | + |
| Pituitary | +++ | ++ |
| Heart | + | +/− |
| Lung | + | +/− |
| Liver | + | + |
| Foetal Liver | + | +/− |
| Kidney | + | + |
| Skeletal Muscle | ++ | ++ |
| Stomach | + | + |
| Intestine | + | + |
| Spleen | + | + |
| Lymphocytes | ++ | ++ |
| Macrophage | − | − |
| Adipose | +++++ | +++++ |
| Pancreas | + | + |
| Prostate | ++ | + |
| Placenta | + | + |
| Cartilage | + | + |
| Bone | − | − |
| Bone Marrow | +/− | +/− |

### Example 2

Analysis of NOT1 Expression in 3T3 Cells

Insulin and dexamethasone induced differentiation of 3T3-L1 cells is an established in vitro model for adipogenesis. Levels of NOT1 mRNA were quantified in this model. The 3T3-L1 fibroblast cell line was obtained from the American Type Culture Collection. Cells were grown to confluence in Dulbeccos modified Eagles Medium containing 10% FCS. Differentiation was undertaken with in Dulbeccos modified Eagles Medium containing 10% FCS supplemented with 0.5 mM isobutyl methyl xanthine, 0.25 uM dexamethasone and insulin (5 ug/ml) for 48 hours followed by Dulbeccos modified Eagles Medium containing 10% FCS supplemented with insulin (5 ug/ml) for a further 48 hours. Cells were grown in Dulbeccos modified Eagles Medium containing 10% FCS for the remainder of the culture period.

On each day of experimentation the cell media was removed by aspiration and the cell monolayer extracted with 1 ml of Trizol. RNA was prepared according to the manufacturer's instructions. Quantification of mRNA transcripts was carried out using the Taqman 5′ nuclease assay as previously described (Wang T & Brown M J (1999) Anal. Biochem. 269 p198-201). Total RNA was treated with DNaseI (Gibco BRL) and reverse transcribed using random hexamers (Stratagene) and Superscript II reverse transcriptase (Gibco BRL). Transcript cDNA levels were then measured using Taqman assay primers and fluorogenic probes as described in example 1. The results are shown in Table 2. In Table 2, T1 refers to day 1, T2 to day 2 etc. and the figures in the right hand column indicate relative levels of Not1 mRNA, the figure on day 1 being 100%.

TABLE 2

| | |
|---|---|
| T1 | 100 |
| T2 | 139 |
| T3 | 57 |
| T4 | 2.8 |
| T5 | 3.7 |
| T6 | 0.9 |
| T7 | 3.9 |
| T8 | 8.7 |
| T9 | 0.6 |
| T10 | n.d. |
| T11 | 3.4 |
| T12 | 8.5 |

The results show that NOT1 expression is downregulated upon differentiation of 3T3-L1 cells, an establised in vitro model for adipogenesis. This finding is consistent with NOT1 having a function in maintaining the undifferentiated pre-adipocyte state.

1. The use of a compound selected from:
(a) a NOT1 or NOT1a polypeptide;
(b) a compound which activates a NOT1 or NOT1a polypeptide; or
(c) a polynucleotide encoding a NOT1 or NOT1a polypeptide, for the manufacture of a medicament for treating:
(i) obesity;
(ii) insulin resistance;
(iii) type 2 diabetes;
(iv) impaired glucose tolerance;
(v) cachexia; or
(vi) liposarcoma.

2. The use according to claim 1 wherein the medicament is used to treat obesity.

3. The use according to claim 1 wherein the medicament is used in the treatment of insulin resistance.

4. The use according to claim 1 wherein the medicament comprises an isolated polypeptide which comprises a polypeptide having at least 95% identity to the NOT1 polypeptide of SEQ ID NO:2 or the NOT1a polypeptide of SEQ ID NO:4.

5. The use according to claim 4 wherein the isolated polypeptide is the NOT1 polypeptide of SEQ ID NO:2 or the NOT1a polypeptide of SEQ ID NO:4.

6. The use according to claim 1 wherein the medicament comprises a compound which activates a NOT or NOT 1a polypeptide.

7. The use according to claim 1 wherein the medicament comprises a polynucleotide encoding a polypeptide having at least 95% identity with the amino acid sequence of SEQ ID NO:2 or SEQ ID NO:4.

8. The use according to claim 7 wherein the polynucleotide comprises a polynucleotide having at least 95% identity with the polynucleotide of SEQ ID NO:1 or SEQ ID NO:3.

9. The use according to claim 7 or 8 wherein the polynucleotide has the polynucleotide sequence of SEQ ID NO:1 or SEQ ID NO:3.


SEQUENCE LISTING (4 sequences)

SEQ ID NO: 1 - DNA, 3427 bp, Homo sapiens
gctcgcgcacggctccgcggtcccttttgcctgtccagccggccgcctgtccctgctccctccctccgtgagtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataaggtataatgggaaatgtttggccatgcttggttgttgcagttcagacaaatgtaacacacacacacatacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaaaaaaaaaa

SEQ ID NO: 2 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 3 - DNA, 1676 bp, Homo sapiens
atgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaa

SEQ ID NO: 4 - protein, 455 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRI